(19) United States
(12) Patent Application Publication
(10) Pub. No.: US 2008/0317822 A1
(43) Pub. Date: Dec. 25, 2008

(54) Methods for Delivery of Nanosome Encapsulated Agents

(75) Inventor: Nazli Azimi, San Juan Caplstrano, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: NuGene

(21) Appl. No.: 11/766,001

(22) Filed: Jun. 20, 2007

(51) Int. Cl. A61K 9/00; A61K 31/70; A61K 38/02; A61P 43/00; A61K 38/18

(52) U.S. Cl. 424/439; 514/12; 514/54; 514/6

(57) ABSTRACT

Vitamins, minerals, antioxidants, and growth factors are encapsulated in nanosomes and the nanosomes suspended in beverages. The nanosomes effectively cross the through the stomach and into the blood stream to be delivered to cells while protecting the contents encapsulated in the cell from degradation prior to delivery to the final target.

### RELATED APPLICATION

This application claims the benefit of and priority to U.S. Utility patent application Ser. No. 11/765,977 filed 20 Jun. 2007, entitled “Personalized Nutrient Deficiency Assay” the contents of which are incorporated by reference herein in its entirety.

### BACKGROUND

The application relates to methods for the delivery of vitamins, minerals, antioxidants, growth factors, amino acids, drugs, and other desirable deliverable to cells via nanosomes suspended in beverages.

### SUMMARY

Vitamins, minerals, antioxidants, and growth factors are encapsulated in nanosomes and the nanosomes suspended in beverages. The nanosomes effectively cross through the stomach and into the blood stream to be delivered to cells while protecting the contents encapsulated in the cell from degradation prior to delivery to the final target.

According to a feature of the present disclosure, a method is disclosed comprising obtaining at least one active ingredient encapsulated in a nanosome and suspending the nanosome in a beverage to be consumed by an organism.

According to a feature of the present disclosure, a composition is disclosed comprising a liquid delivery medium, a plurality of nanosomes suspended in the liquid delivery medium, and at least an active ingredient contained within at least one nanosome.

### DRAWINGS

The above-mentioned features and objects of the present disclosure will become more apparent with reference to the following description taken in conjunction with the accompanying drawings wherein like reference numerals denote like elements and in which:

FIG. 1 is a cross-sectional view of an embodiment of a nanosome having at least one biologically active ingredient; and

FIG. 2 is a perspective view of an embodiment of a beverage having the nanosomes containing active ingredients.

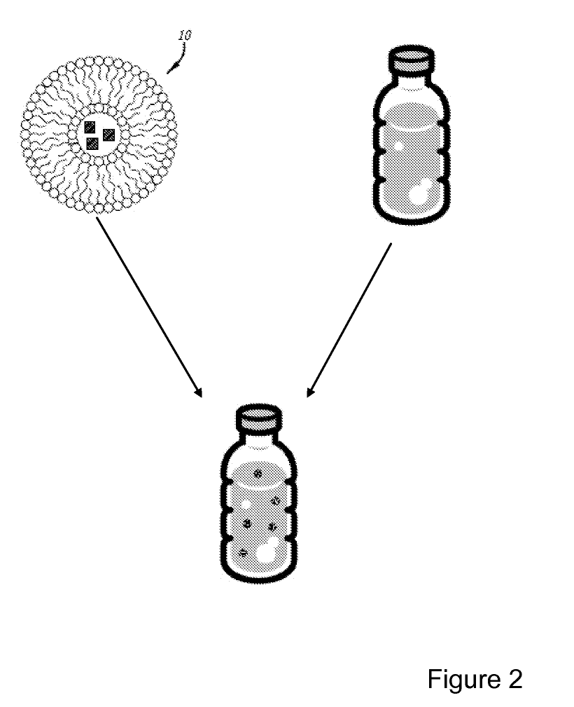

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

As used in the present disclosure, the term “active ingredient” shall be defined as a component of a nutraceutical that interacts with or effects changes in tissues, cells, biological pathways, or native molecules. For example, active ingredients may comprise pharmaceutical agents, vitamins, minerals, hormones, metabolites, amino acids, nucleic acids, antioxidants, growth factors and other proteins.

As used in the present disclosure, the term “nutraceutical” shall be defined as a ingested substance having at least one active ingredient designed to improve the health of an organism.

According to embodiments, the uptake of nutrients is improved by packaging the nutrients into nanosomes, which are readily absorbed through the skin and the contents thereof delivered intracellularly. By encapsulating nutrients, growth factors, etc., into nanosomes, they may be absorbed through the digestive tract without degradation and into the target cells. Similarly, fat soluble nutrients and growth factors are storable and deliverable using nanosomes as well.

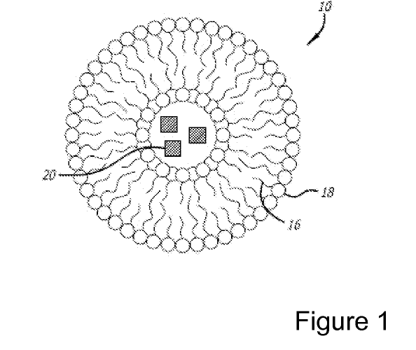

According to embodiments illustrated in FIG. 1, nanosomes 10, which are essentially small (˜30 nm) liposomes, are formed from a bilayer of molecules having hydrophobic head 18 and hydrophilic tail 16. For example, a nanosome may be constructed having a phospholipid and cholesterol bilayer. Like cells, the phospholipids aggregate together to form a “capsule” with the hydrophobic portion of the bilayer on the outside and the hydrophilic portion on the inside, much as oil micelles form in water. Encapsulated in nanosome 10 are one or more active ingredients 20 to be delivered.

Nanosomes may be desirable, according to embodiments, because they improve bioavailability of the active ingredients encapsulated within them. Bioavailability is increased because more active ingredient is actually delivered to its target. Moreover, nanosomes readily cross through the gastrointestinal membrane, thereby effectively transporting the active ingredients into the blood stream. Additionally, because the bioavailability is increased, less active ingredient must be used, which reduces the costs associated with producing and delivering active ingredients to effect a result. Because the active ingredients encapsulated in the nanosome are protected from the environment, the active ingredients have a longer shelf life and are more stable on average than those not encapsulated. Importantly, active ingredients encapsulated within a nanosome can undergo pasteurization without destroying the active ingredient in the process.

Most importantly, however, the nanosome protects the active ingredients from oxidation, hydrolysis, reduction, and enzymatic reaction, thereby allowing the active ingredient to arrive at its target in an active state. Finally, nanosome encapsulation masks odor and taste of the active ingredients, making them more pleasing to those who use the active ingredients. Therefore, nanosome containing active ingredients may be suspended in beverages with no effect to the taste, smell, or consistency of the beverage itself.

The growth factors of the present disclosure may be encapsulated in nanosomes, and the nanosomes put into a delivery vehicle. Thus, they will be effectively delivered through the skin or into the blood stream via the gastrointestinal tract. Thereafter, the nanosomes may be absorbed into cells by fusing with cellular membranes or via endocytosis, for example.

Nanosomes are excellent tools for delivering pharmacological active ingredients through the skin or the digestive tract directly to the cell without exposing the active ingredients to degradation events prior to delivery. Additionally, nanosomes allow active ingredients that would otherwise only inefficiently arrive at the target cells, for example, water soluble active ingredient applied topically. Thus, in combination, with the novel active ingredients disclosed herein, nanosomes provide a delivery device to efficiently cause the active ingredients to be delivered intracellularly.

The active ingredients of the present disclosure may be encapsulated in nanosomes, and the nanosomes put into a liquid delivery vehicle. Thus, they will be effectively delivered through the blood stream. Thereafter, the nanosomes may be absorbed into cells by fusing with cellular membranes or via endocytosis, for example.

The liquid delivery vehicle may comprise soft drinks, juices, drinking water, and nearly any other beverage consumable by humans and animals. Moreover, because nanosomes are able to be pasteurized without degradation and provided the active ingredients are not substantially heat sensitive, the nanosomes may be suspended in milk and soups, for example. Coffee, tea, and alcoholic beverages are also contemplated as beverage delivery vehicles of the present disclosure.

As such, the nanosome encapsulated nutraceuticals of the present disclosure, provide an effective platform to children and pets. Children, in particular often object to the consistency and flavor of medications or vitamins and refuse to take them. Nanosomes masks odors and flavors thereby making the active ingredients more palatable.

According to embodiments, the active ingredients of the present disclosure are encapsulated in nanosomes and disposed in a variety of liquid delivery vehicles, as disclosed herein. For example, any of copper peptides; growth factors including EFG, KGF, TGF-β3, thymosin β4, IGF-1, aFGF, bFGF, VEGF; peptide sequences that mimic or enhance the activity of proteins, proteases; thioredoxin, arbutin, coenxyme Q10; licorice extract, oryza, sative hull extract; green tea extract; kojic acid; glycolic acid; hyaluronic acid; vitamins; minerals; mucleic acids; and amino acids are encapsulated into nanosomes, and nanosomes 300 put into delivery vehicles 402 for direct delivery to the areas affected by or predisposed to hair loss.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
obtaining at least one active ingredient encapsulated in a nanosome;
suspending the nanosome in a beverage to be consumed by an organism.

2. The method of claim 1, wherein the at least one active ingredient is at least one vitamin, mineral, amino acid, growth factor, or antioxidant.

3. The method of claim 2, wherein the growth factors comprise at least one of TGF-β3, hyaluronic acid, aFGF, KGF, TGF-β3, TRX, and copper peptide.

4. The method of claim 3, wherein the copper peptide is at least one of the copper containing peptides selected from the group consisting of: Cu(II)-His-Val-His, His-Ala-His-Cu(II), and Gly-His-Lys-Cu(II).

5. The method of claim 1, wherein the beverage is a carbonated soft drink.

6. The method of claim 1, wherein the beverage is a vegetable or fruit juice-based drink.

7. The method of claim 1, wherein the beverage is drinking water.

8. The method of claim 1, wherein the beverage is one of: an alcohol-based drink, coffee, tea, and cocoa.

9. The method of claim 1, wherein suspension of the nanosomes is performed in a water supply.

10. The method of claim 1, wherein the beverage is used to deliver a pharmacologically active substance.

11. The method of claim 1, wherein the beverage comprises a delivery vehicle to provide medications to children.

12. A composition comprising:
a liquid delivery medium;
a plurality of nanosomes suspended in the liquid delivery medium; and
at least an active ingredient contained within at last one nanosome.

13. The composition of claim 12, wherein the liquid delivery medium is a soft drink.

14. The composition of claim 12, wherein the liquid delivery medium is a vegetable or fruit juice-based drink.

15. The composition of claim 12, wherein the liquid delivery medium is selected from a group consisting of: coffee, tea, cocoa, and alcoholic beverages.

16. The composition of claim 12, wherein the liquid delivery medium comprises drinking water.

17. The composition of claim 12, wherein the liquid delivery medium is converted to a solid prior to being consumed by an organism.

18. The composition of claim 12, wherein the active ingredient comprises at least one growth factor.

19. The composition of claim 18, wherein the at least one growth factor comprises at least one of TGF-β3, hyaluronic acid, aFGF, KGF, TGF-β3, TRX, and copper peptide.

20. The method of claim 19, wherein the copper peptide is at least one of the copper containing peptides selected from the group consisting of: Cu(II)-His-Val-His, His-Ala-His-Cu(II), and Gly-His-Lys-Cu(II).
